(19) United States
(12) Patent Application Publication
(10) Pub. No.: US 2019/0239794 A1
(43) Pub. Date: Aug. 8, 2019

(54) ELECTROENCEPHALOGRAM DETECTING DEVICE AND PROGRAM

(71) Applicant: University of Tsukuba, Ibaraki (JP)

(72) Inventors: Masayuki Ide, Ibaraki (JP); Masahiro Kawasaki, Ibaraki (JP); Eri Miyauchi, Ibaraki (JP); Keiichi Kitajo, Saitama (JP)

(21) Appl. No.: 16/339,225

(22) PCT Filed: Oct. 10, 2017
(86) PCT No.: PCT/JP2017/036699

§ 371 Date: Apr. 3, 2019

(30) Foreign Application Priority Data
Oct. 7, 2016 (JP) 2016-199492

(51) Int. Cl. A61B 5/16; A61B 5/0484; A61B 5/00

(52) U.S. Cl. CPC A61B 5/165; A61B 5/4088; A61B 5/0484

(57) ABSTRACT

The objective of the present invention is to provide an electroencephalogram detecting device and program for evaluating correlations between a plurality of regions in a brain, on the basis of reactions of the regions of the brain to an electromagnetic stimulus applied to the brain. The electroencephalogram detecting device includes a signal generating unit configured to apply an electromagnetic stimulus to a prescribed region in a brain of a subject, an electroencephalogram detecting unit including a plurality of electrodes for detecting electroencephalograms in a plurality of regions in the brain in which the electromagnetic stimulus was applied to the prescribed region, and a computing unit configured to evaluate correlations between a part of the brain associated with the prescribed region to which the electromagnetic stimulus was applied and parts of the brain associated with the plurality of regions, on the basis of a plurality of electroencephalograms obtained respectively from the plurality of electrodes.

### TECHNICAL FIELD

The present invention relates to an electroencephalogram detecting device and program for detecting electroencephalograms. Priority is claimed on Japanese Patent Application No. 2016-199492, filed Oct. 7, 2016, the content of which is incorporated herein by reference.

### BACKGROUND ART

The biological basis and mechanism of neuropsychiatric disorders such as depression, schizophrenia, bipolar disorder, and dementia have not yet been elucidated and diagnosis of these disorders is performed according to criteria for clinical symptoms and inquiries (for example, diagnosis criteria DSM-5 for mental disorders, depression inquiry scale MADRS, and the like) and the objectivity thereof is not sufficient. Accordingly, it is necessary to establish more objective biological diagnostic markers for diagnosis of these neuropsychiatric disorders and appropriate treatment selection.

For the purpose thereof, various modalities have been proposed as objective test marker candidates that reflect a pathological condition of neuropsychiatric disorder such as depression and their evaluation means. For example, the modalities are an electroencephalogram (EEG), magnetoencephalography (MEG), a transcranial magnetic stimulation-induced electroencephalogram (TMS-EEG), positron CT (PET), nuclear magnetic resonance imaging (MRI), near-infrared light imaging, and the like.

Among these modalities, a transcranial magnetic stimulation-induced electroencephalogram can evaluate a brain function and response in a case that a quantitative stimulus is applied to the brain with the time resolution associated with brain activity and is promising in the viewpoint of excellent convenience of clinical application (Non-Patent Literature 5 and 7). A transcranial magnetic stimulation-induced electroencephalogram (hereinafter referred to as TMS-EEG) is used for a method of applying a local eddy current to a surface of the brain according to a change in a magnetic field and evaluating reactions of a plurality of regions of the brain with respect to an electromagnetic stimulus through EEG measurement.

On the other hand, a relation between parts of the brain has been determined to be important for the evaluation of a brain function and the evaluation of a pathological condition (Non-Patent Literature 1, 2, 3, and 4). Furthermore, the evaluation of synchrony and a phase difference between electroencephalograms of parts is also known to be important regarding the relation between the parts of the brain (Non-Patent Literature 1, 2, and 6).

### CITATION LIST

### Non-Patent Literature

### [Non-Patent Literature 1]

J. P. Lachaux, E. Rodriguez, J. Martinerie, F. J. Varela, Measuring phase synchrony in brain signals, Human Brain Mapping Volume 8, Issue 4 1999 194-208.

### [Non-Patent Literature 2]

M. Kawasaki, K. Kitajo, Y. Yamaguchi, Fronto-parietal and fronto-temporal theta phase synchronization for visual and auditory-verbal working memory, Frontiers in Psychology, Published online, 18 Mar. 2014.

### [Non-Patent Literature 3]

Alexander A. Fingelkurts and Andrew A. Fingerkurts, Altered Structure of Dynamical Electroencephalogram Oscillatory Pattern in Major Depression, biopsych, 11 Dec. 2014.

### [Non-Patent Literature 4]

Yuezhi Li, Cheng Kang, Xingda Qu, Yunfei Zhou, Wuyi Wang and Yong Hu, Depression-Related Brain Connectivity Analyzed by EEG Event-Related Phase Synchrony Measure, Frontiers in Psychology, Published online, 26 Sep. 2016.

### [Non-Patent Literature 5]

Faranak Farzan, Mera S. Barr, Paul B. Fitzgerald and Zafiris J. Daskalakis, Combination of TMS with EMG & EEG Application in Diagnosis of Neuropsychiatric Disorders, InTech, EMG Methods for Evaluating Muscle and Nerve Function, Chapter 18, Published online, 11 Jan. 2012.

### [Non-Patent Literature 6]

Arj an Hillebrand, Prejaas Tewarie, Edwin van Dellen, Meichen Yu, Ellen WS Carbo, Linda Douw, Alida A. Gouw, Elisabeth CW van Straaten, and Cornelis J. Stam, Direction of information flow in large-scale resting state network is frequency-dependent, PNAS, vol. 113, no. 14, pp. 3867-3872, Published online, 5 Apr. 2016

### [Non-Patent Literature 7]

Masahiro Kawasaki, Yutaka Uno, Jumpei Mori, Kenji Kobata, and Keiichi Kitajo, Transcranial magnetic stimulation-induced global propagation of transient reset associated with directional information flow, Frontiers in Human Neuroscience, 25 Mar. 2014

### SUMMARY OF INVENTION

### Technical Problem

Although a method of evaluating reactions of a plurality of regions of the brain with respect to an electromagnetic stimulus applied to a locally fixed part of the brain according to electroencephalogram measurement is disclosed in the TMS-EEG techniques disclosed in Non-Patent Literature 5 and 7, there is a problem in that they are not suitable for the pathological condition evaluation and determination of neuropsychiatric disorders as a means for evaluating the electroencephalogram synchronization and relation between a plurality of regions of the brain determined to be important for the pathological condition evaluation of neuropsychiatric disorders because the detection of the phase synchrony based on an electroencephalogram phase difference is not taught.

Although a method based on a brain signal phase difference between regions of the brain is disclosed in measurement techniques based on EEG MEG and the like disclosed in Non-patent Literature 1, 2, and 6, there is a problem in that these are not suitable for the pathological condition evaluation and determination of neuropsychiatric disorders because the phase synchronization induced by TMS is not taught.

The present invention provides an electroencephalogram detecting device and program for evaluating a correlation between regions and evaluating pathological conditions of neuropsychiatric disorders on the basis of reactions of a plurality of regions of a brain with respect to an electromagnetic stimulus applied to a prescribed region of the brain.

### Solution to Problem

According to the present invention, an electroencephalogram detecting device includes a signal generating unit configured to apply an electromagnetic stimulus to a prescribed region in a brain of a subject; an electroencephalogram detecting unit including a plurality of electrodes for detecting electroencephalograms in a plurality of regions in the brain in which the electromagnetic stimulus was applied to the prescribed region; and a computing unit configured to evaluate correlations between a part of the brain associated with the prescribed region to which the electromagnetic stimulus was applied and parts of the brain associated with the plurality of regions, on the basis of a plurality of electroencephalograms obtained respectively from the plurality of electrodes.

According to the present invention, in the above-described configuration, it is possible to evaluate the correlations between the regions in a case that the electroencephalogram detecting unit detects the electroencephalograms in the plurality of regions with respect to a reaction of the brain to the electromagnetic stimulus applied to the prescribed region of the brain by the signal generating unit.

In the electroencephalogram detecting device of the present invention, the electroencephalogram detecting unit detects the electroencephalograms in the plurality of regions with respect to a reaction of the brain to the electromagnetic stimulus applied to the prescribed region of the brain by the signal generating unit and phase synchrony based on a phase difference between the electroencephalograms of any two regions included in the plurality of regions is evaluated, such that a correlation between any two regions may be evaluated.

In the electroencephalogram detecting device of the present invention, the computing unit may compute phase synchrony between two points in the prescribed region to which the electromagnetic stimulus was applied and one region among the plurality of regions and index a relation of corresponding parts of the brain between the two points on the basis of the phase synchrony.

According to the present invention, in the above-described configuration, it is possible to index the reaction of the brain to which the electromagnetic stimulus was applied and quantitatively measure a function of the brain according to the phase synchrony computed by the computing unit.

In the electroencephalogram detecting device of the present invention, the signal generating unit may apply the electromagnetic stimulus to a portion of a visual area of the brain and the computing unit may index a relation between parts of the brain in the visual area and a motor area.

According to the present invention, a program causes a computer to: generate an electromagnetic stimulus to be applied to a prescribed region in a brain of a subject; detect electroencephalograms in a plurality of regions of the brain using a plurality of electrodes disposed on the brain to which the electromagnetic stimulus was applied; and compute correlations between a part of the brain associated with the prescribed region to which the electromagnetic stimulus was applied and parts of the brain associated with the plurality of regions, on the basis of a plurality of electroencephalograms obtained respectively from the plurality of electrodes.

### Advantageous Effects of Invention

According to the present invention, an electroencephalogram detecting device can evaluate pathological conditions of neuropsychiatric disorders by evaluating a correlation between regions on the basis of reactions of a plurality of regions of a brain with respect to an electromagnetic stimulus applied to a prescribed region of the brain.

### BRIEF DESCRIPTION OF DRAWINGS

FIG. 1 is a block diagram showing a configuration of an electroencephalogram detecting device 1.

FIG. 2 is a diagram showing an example of a specific configuration of the electroencephalogram detecting device 1.

FIG. 3 shows display of a screen used in a trial for WM.

FIG. 4 is a diagram showing a detection result between electrodes E detected by the trial.

FIG. 5 is a graph showing an average counted number of electrode pairs indicating a prescribed value in the trial.

FIG. 6 shows a biphasic stimulator for applying TMS.

FIG. 7 is a diagram showing a result of performing ECT on a subject.

FIG. 8 is a diagram showing a result of performing ECT on a subject.

FIG. 9 is a diagram showing a result of performing ECT on a subject.

### DESCRIPTION OF EMBODIMENTS

Hereinafter, an electroencephalogram detecting device according to an embodiment will be described with reference to the drawings.

### [Device Configuration]

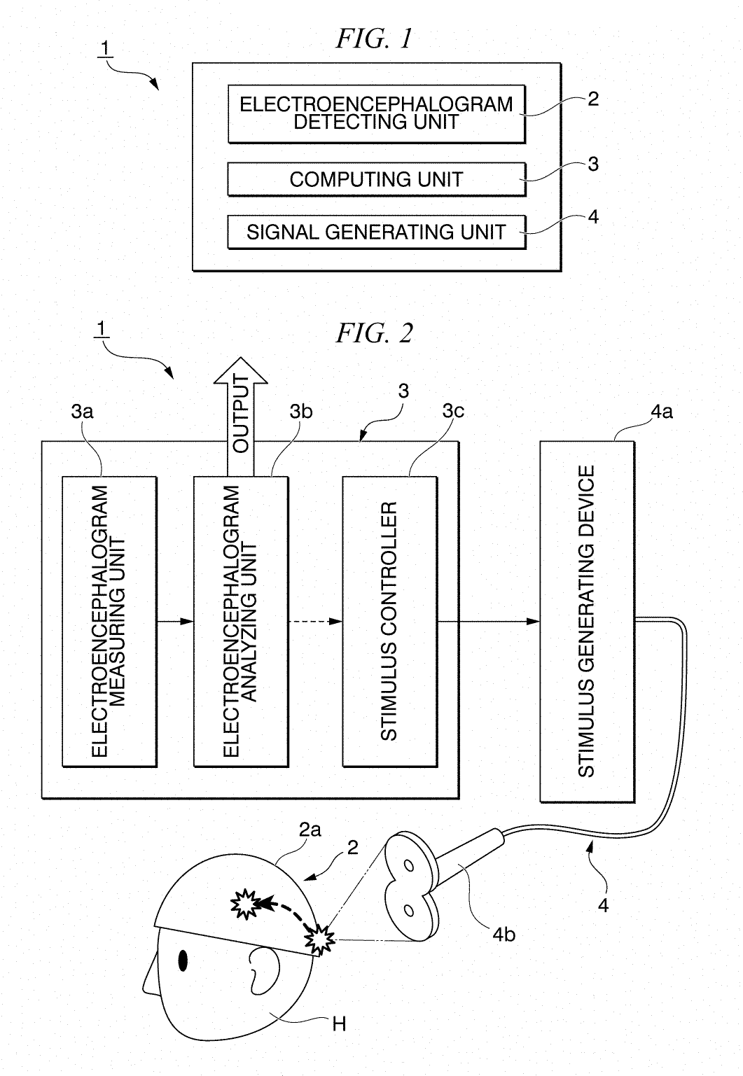

FIG. 1 is a block diagram showing an example of a configuration of an electroencephalogram detecting device 1. The electroencephalogram detecting device 1 includes, for example, an electroencephalogram detecting unit 2, a computing unit 3, and a signal generating unit 4.

FIG. 2 is a diagram showing an example of a specific configuration of the electroencephalogram detecting device 1. The electroencephalogram detecting unit 2 includes, for example, a cap unit 2a formed to cover a head portion of a subject H. A plurality of electrodes for detecting electroencephalograms in a plurality of regions of the head portion of the subject H are disposed inside the cap unit 2a. The electroencephalogram detecting unit 2 detects the electroencephalograms in the plurality of regions in a brain of the subject H by providing the plurality of electrodes.

The signal generating unit 4 applies, for example, a local eddy current (TMS) to the surface of the brain of the subject H. The signal generating unit 4 includes, for example, a stimulus generating device 4a configured to generate a TMS signal and a coil unit 4b configured to cause a TMS electromagnetic pulse to be generated on the basis of the signal generated by the stimulus generating device 4a. The coil unit 4b is formed in, for example, a figure-of-eight coil shape.

For example, the computing unit 3 evaluates correlations between a part of the brain associated with the prescribed region to which the electromagnetic stimulus was applied and parts of the brain associated with the plurality of regions, on the basis of a plurality of electroencephalograms obtained respectively from the plurality of electrodes of the electroencephalogram detecting unit 2.

For example, the computing unit 3 includes an electroencephalogram measuring unit 3a configured to acquire the electroencephalograms from the plurality of electrodes of the electroencephalogram detecting unit 2, a waveform analyzing unit 3b configured to analyze waveforms of the electroencephalograms measured by the electroencephalogram measuring unit 3a and output analysis results, and a stimulus controller 3c configured to control the stimulus generating device 4a.

For example, the waveform analyzing unit 3b computes phase synchrony of an electroencephalogram waveform between two points in the prescribed region to which the electromagnetic stimulus was applied and one region among the plurality of regions of the brain measured by the electroencephalogram measuring unit 3a. The phase synchrony will be described below.

For example, the waveform analyzing unit 3b indexes a relation of corresponding parts of the brain between the above-described two points in the brain on the basis of the computed phase synchrony. The stimulus controller 3c controls the stimulus generating device 4a such that TMS to be applied to the brain surface of the subject H is generated from the coil unit 4b.

Some or all of the electroencephalogram measuring unit 3a, the waveform analyzing unit 3b, and the stimulus controller 3c are implemented, for example, in a case that a hardware processor such as a central processing unit (CPU) executes a program (software). Also, some or all of these components may be implemented by hardware (a circuit unit including circuitry) such as large scale integration (LSI), an application specific integrated circuit (ASIC), a field-programmable gate array (FPGA), or a graphics processing unit (GPU) or may be implemented by cooperation between software and hardware.

In order to construct an index for determining the severity of a neuropsychiatric disorder such as depression which is not possible in conventional TMS-EEG techniques, in the above-described device configuration, a system for recruiting subjects who are healthy people and evaluating and determining a function of the working memory that plays an important role in information processing for basic abilities of hearing, vision, language, and the like in the brain using EEG phase synchronization between different brain parts in TMS-EEG as an index is first constructed.

This is because working memory abnormalities are thought to be deeply related to neuropsychiatric disorders such as depression, schizophrenia, bipolar disorder and dementia. In order to evaluate the working memory function, subjects were asked to perform tasks (WM tasks) that are thought to use the working memory and TMS-EEG phase synchronization in a process thereof was evaluated.

### 1. INTRODUCTION

In terms of the evaluation experiment of a relation between parts in charge of the working memory which is the basic function of the brain using healthy people

Conventionally, a theory in which phase synchronization of global theta waves between a frontal area and a sensory area of the brain is connected between a plurality of brain regions related to an executive process of working memory (WM) has been proposed. However, little is known regarding the directionality of a connection network related to the interaction between the regions in the brain (i.e., a direction-dependent relationship such as top-down from the frontal area to a visual area or bottom-up from the visual area to the frontal area).

Brain neural substrates of working memory (WM) are thought to include a plurality of separate systems. That is, an executive system is positioned in a prefrontal area and is thought to include different systems including a posterior sensory area for the maintenance system, a parietal area for visual WM, and a temporal area for auditory WM. Recent human EEG studies have shown that a global network of the brain with large-scale phase synchronization has an important role in WM. Specifically, theta wave rhythms in distributed brain regions are thought to interact with each other. Moreover, it has been suggested that low-frequency synchronizations connect the frontal area with posterior sensory areas in relation to an executive system function.

However, network directionality of such interactions in WM is not clear. That is, directionality of either a signal (bottom-up) from the sensory area to the frontal area or a signal (top-down) from the frontal area to the sensory area is not clear. Previous studies based on transcranial magnetic stimulation (TMS) and an EEG have suggested that a stimulus of a specific neural region of the brain based on single-pulse TMS can manipulate local synchronization and induce spatial propagation of synchronization of the EEG during a resting state.

Therefore, it is plausible that this method could identify network directionality among WM-relevant brain regions by focusing on TMS-induced changes in EEG rhythms during WM tasks. For example, if phase synchronization changes in a case that TMS is delivered to the frontal cortex, directionality is likely to be top-down. In contrast, if phase synchronization changes in a case that TMS is delivered to the sensory cortex, directionality is likely to be bottom-up. Hence, this study aims to clarify WM network directionality.

In the experiment, two types of WM manipulation tasks [an auditory WM task (AWM) and a visual WM task (VWM)] were performed and single-pulse TMS was delivered to three target areas (the frontal area, the visual area, and the auditory area) of the brain in the two tasks. The tasks were also performed in a sham-TMS condition and a no-TMS condition.

### 2. METHODS

### 2.1. Participants

Ten healthy right-handed volunteers (four females; mean age=23.5±1.1 years, range 20 to 33 years) with normal or corrected-to-normal visual acuity, normal hearing acuity, and normal motor performance took part in this EEG experiment. All participants gave written informed consent, and the protocol was approved by the Ethical Committee of the RIKEN (in accordance with the Declaration of Helsinki), before the experiments were performed.

### 2.2. Auditory Working Memory Task

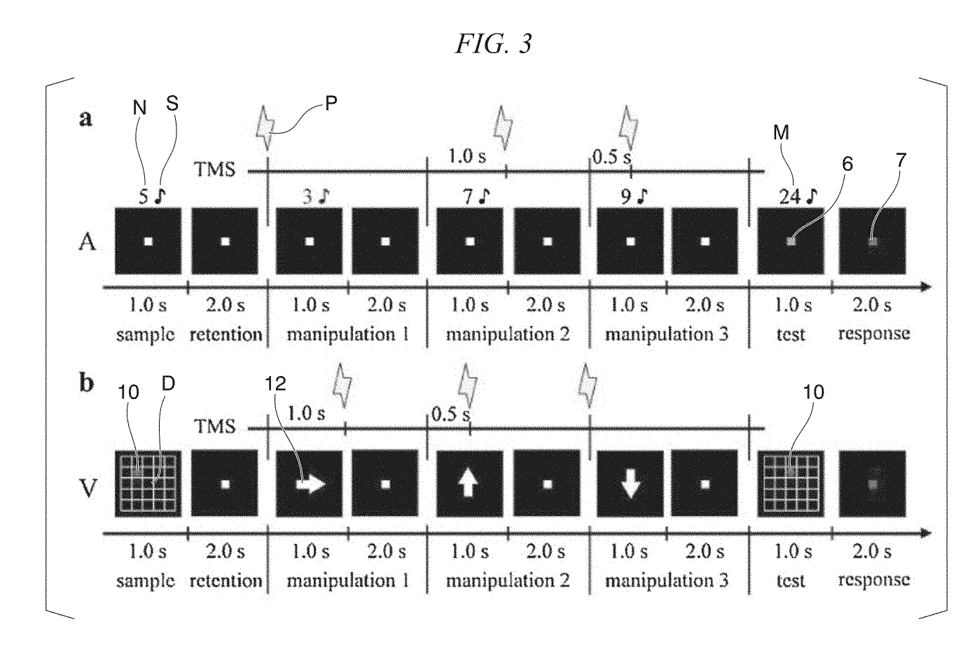

FIG. 3 is a diagram showing display of a screen used for a trial for the WM. In the auditory trial, the trial was performed in a state in which participants, wearing earphones, faced a computer screen placed 60 cm away. At the beginning of each trial, participants were required to memorize a 1-digit number N presented as an auditory stimulus through the earphones for 1 s (see FIG. 3(a)). After a 2-s retention interval, another 1-digit number N was presented as an auditory stimulus for 1 sec, and participants were asked to add the presented number N to the earlier memorized number N.

This mental addition (“manipulation phase”) was repeated 3 times, and a probe number was presented as an auditory stimulus, after a white fixation point 5 had turned to a gray fixation point 6 (test display). Participants had to determine whether the probe number matched the mental calculation total M within 2 s (in a case that a red fixation point 7 was reached) by pressing a button. Inter-trial interval (ITI) duration was set to 2 s. Stimuli were generated using Matlab 2010 (registered trademark) with the psychophysics toolbox extension.

### 2.3. Visual Working Memory Task

As shown in FIG. 3(b), at the beginning of each trial with respect to visual sensation, a 5×5 square grid D and a 1×1 red circle 10 were displayed on a screen D of a computer for 1 sec. Participants were required to memorize the position of the red circle 10 displayed on the screen D. After a 2-s retention interval, participants needed to mentally move the red circle 10 within the grid D in accordance with a white arrow 12 presented at the center of the screen D for 1 s (“manipulation phase”). The arrow 12 was directed upward, downward, rightward, or leftward.

Participants were asked to repeat this mental manipulation 3 times. Thereafter, the red circle 10 was shown on the display to indicate whether a mentally determined position within the grid D of the red circle 10 matched a visual probe (test display). The button pressed in the experiment, the duration of the ITI, and the creation of the stimuli were similar to those of the AWM task.

### 2.4. TMS

In each trial, three pulses of single-pulse TMS from the coil unit 4b were applied to frontal (Fz), temporal (TP7), or parietal (Pz) areas during the manipulation phase of the task. Specifically, for each manipulation cue (a number with a note symbol S in FIG. 3(a) for the AWM tasks or a white arrow 12 in FIG. 3(b) for the VMM tasks), a single pulse P of TMS was applied as one of three cue-TMS stimuli that started at asynchronous timings (0, 500, and 1000 ms).

In the experiment, a figure-of-eight coil, with a 70-mm ring diameter connected to a biphasic stimulator (the signal generating unit 4) (Magstim Rapid, Magstim Company Ltd., UK: see FIG. 6) was used for TMS application. For maintaining the coil position and direction throughout each session, the flexible arm of a camera stand was used. Prior to performing the experiments, the TMS intensity of each participant was determined as the 95% motor threshold which was a minimum intensity making his/her index finger twitch. To test placebo effects of TMS, the sham-TMS condition was used by applying TMS pulses P to a location 15 cm from the top of the head.

### 2.5. Experimental Procedure

Each participant completed ten separate sessions. The ten separate sessions included 2 WM tasks (AWM and VWM tasks)×5 TMS conditions (frontal, temporal, parietal, sham, and no-TMS) in a counterbalanced order. Each session consisted of 24 trials (72 TMS applications). All participants were well trained before the EEG-measurement sessions.

### 2.6. EEG Recordings

EEG recordings were performed using 67 [ch] scalp electrodes (Ag/AgCl), embedded in a TMS EEG electrode cap (the electroencephalogram detecting unit 2) (EASYCAP Gmbh, Germany), and in accordance with placement based on the international 10/10 system. A sampling rate was 1000 [Hz]. Reference electrodes were disposed on the right and left mastoids. Electrode impedance was kept below 10 [kΩ]. Also, scalp electrodes (total 4 [ch]) placed vertically and horizontally from the right and left eyes were used for recording electrooculography (EOG). EEG signals were amplified and recorded using an electroencephalograph (Brainamp MR+ Brain Products, Germany).

### 2.7. EEG Data Preprocessing

In the experiment, EEG data acquired by the electroencephalogram detecting unit 2 from the subject H was analyzed by the computing unit 3 implemented by, for example, a computer. EEG data was segmented to 3-s epochs for the manipulation period from the instruction onset for manipulation. In the analysis, linear interpolation was used and EEG data points affected by TMS artifacts (from −1 to 7 ms post-TMS onset) were removed. The EEG epochs were subjected to info-max independent components analysis (ICA).

ICA components that were significantly correlated with the vertical or horizontal EOGs were eliminated as ocular artifacts. ICA-corrected data was recalculated using regression for the remaining components. To eliminate volume conduction errors, current source density analysis at each electrode position was performed and the spherical Laplace operator was applied to the voltage distribution on the scalp surface.

### 2.8. Wavelet Analysis

Hereinafter, a process of analyzing an electroencephalogram in the computing unit 3 will be described.

In the analysis, wavelet transforms using Morlet's wavelet function was applied. Six time points were selected for the analysis (0, 500, 1000, 1500, 2000, and 2500 ms). The phase for each time point at each TMS application was the arctangent of the original, convoluted EEG signal s(t) resulting from a complex Morlet's wavelet function w(T, F).

[Math. 1]

w(t,f)=(σₜ\sqrt{π})^{-1/2} exp(−t²/2σₜ²)exp(i2πft) (1)

Here, σₜ is the standard deviation of the Gaussian window. The wavelets used are characterized by a constant ratio (f/σ_{f}=7), with f ranging from 2 to 20 hertz (steps of 1 [Hz]). In order to index a phase relation between any two electrodes, phase synchrony (a phase locking value (PLV)) is calculated at time (t) and frequency (F) as follows.

$$\begin{matrix}[ Math . 2 ] & \\ PLV ( t ) = \frac{1}{N} \vert {\sum }_{n = 1}^{N} {e}^{i {Δθ}_{jk} ( t , f , n )} \vert & ( 2 )\end{matrix}$$

Here, Δθⱼk(t, f, n) is a phase difference between j^{th} and k^{th} electrodes. For example, N is, the number of trials to be analyzed. For example, N=20 [times]. n is the index of each trial. In the experiment, the PLV for each subject was first calculated and the PLV at each time point for manipulation periods was compared with the averaged PLV for baseline periods (i.e., ITI), by using the Wilcoxon sign rank test with Bonferroni correction.

In analysis, region-of-interest (ROI) analysis was made and Fz, TP7, and Pz were selected as the representative frontal, temporal, and parietal electrodes in reference to previous studies of the inventors of the present application. The PLV between these three ROI electrodes and the other electrodes was evaluated.

### 3. RESULTS

### 3.1. Behavioral Results

Subject-averaged accuracy rates (±s.d.) during the AWM were as follows: 96.7±1.3, 96.0±0.8, 97.2±0.6, 96.3±1.0, and 96.5±1.2% for no, frontal, temporal, parietal, and sham-TMS conditions, respectively. Subject-averaged accuracy rates (±s.d.) during the VWM were as follows: 96.9±1.3, 95.6±1.3, 96.0±1.1, 96.3±1.5, and 95.6±0.9% for no, frontal, temporal, parietal, and sham-TMS conditions, respectively. A 2-factor ANOVA revealed no main effect of task (F1, 90=0.42 and p=0.52), TMS conditions (F4, 90=0.26 and p=0.90), and no significant interaction (F4, 90=0.14 and p=0.97). These results indicated that EEG comparisons among the different conditions were not influenced by either task difficulty or TMS effects.

### 3.2. EEG Results

It was identified that electrode pairs showing the PLV at each time point were significantly higher than the averaged PLV for the base line period (p<0.05; Bonferroni correction) on the basis of the results. Because the previous study investigated theta synchronization modulation, the inventors of the present application focused on the theta-range (e.g., 4 Hz) PLV between the frontal and other electrodes, between the temporal and other electrodes, and between the parietal and other electrodes.

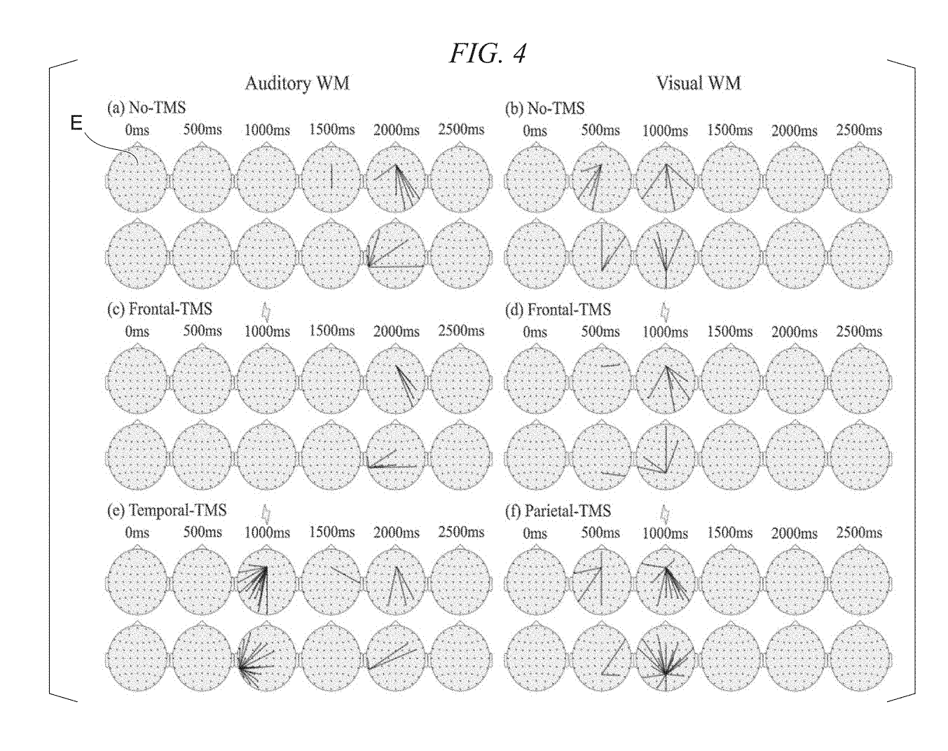

FIG. 4 is a diagram showing detection results between the electrodes E detected by the trial. As shown, in the brain receiving electromagnetic stimulation, the electroencephalogram detecting unit 2 detects electroencephalograms in a plurality of regions of the brain. As shown, significant pairs between the ROI electrodes E and other electrodes E at each time point during the no-TMS, frontal TMS, and temporal TMS (parietal TMS) conditions during the AWM (VWM) tasks are shown. As shown, the 1000 [ms]-TMS results are shown as the representative results.

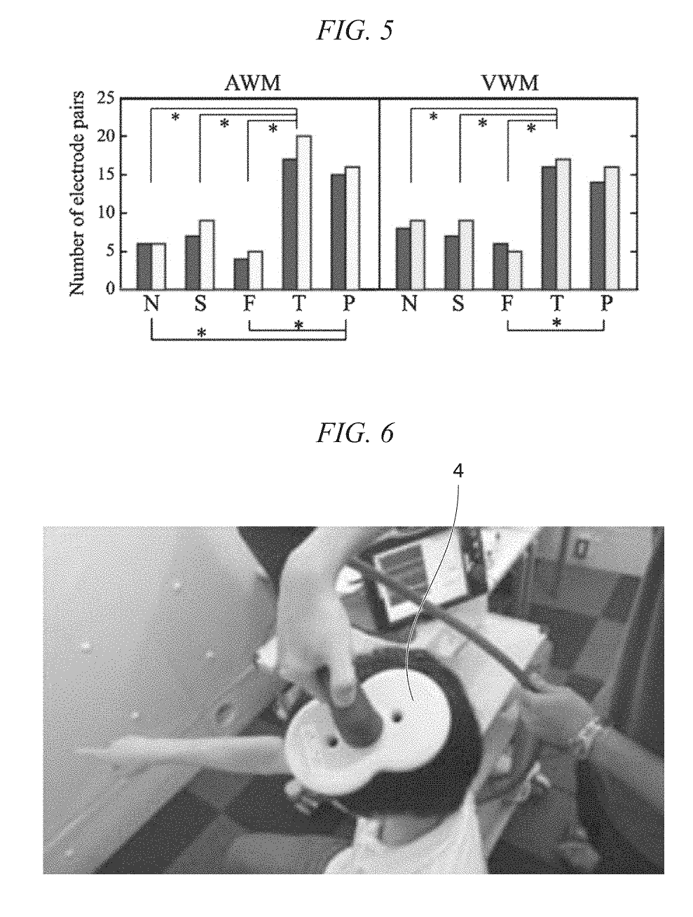

FIG. 5 is a graph showing an average counted number of electrode pairs indicating a prescribed value in the trial. As shown, the average counted number of electrode pairs showing a significantly higher theta (4 Hz) PLV for manipulation periods than that for ITI periods (p<0.05; Bonferroni correction) among 6 latencies (0 ms, 500 ms, 1000 ms, 1500 ms, 2000 ms, and 2500 ms) and among 3 TMS application timings (0 ms, 500 ms, and 1000 ms) is shown.

The no-TMS condition included several significant pairs: between the frontal and other electrodes and between the temporal (parietal) and other electrodes during the AWM (VWM) tasks. These results were similar to those from the frontal-TMS and sham-TMS conditions. It was shown that sensory area-TMS (i.e., temporal-TMS and parietal-TMS) conditions significantly increased the number of significant pairs between the frontal and other electrodes and between the TMS-targeted and other electrodes E in comparison with the no-TMS, frontal-TMS, and sham-TMS conditions (p<0.05; Chi squared test with Bonferroni corrected multiple comparison).

This trend (the number of significant pairs) was almost the same for TMS application times. The above results for the analyses using the single time point of EEG data might be sensitive to noise or extreme points. Therefore, the analyses need to be redone averaging over longer time windows than a single time point. The PLV data was averaged over 100 msec time windows; −50 [ms] to 50 [ms] from the onset of the TMS application and the same statistical analyses were conducted under all the conditions.

As the results, the number of electrodes E showing significant connectivity was 0 (from the frontal electrode) and 0 (from the temporal electrode) under no-TMS, 0 (from the frontal electrode) and 0 (from the temporal electrode) under frontal-TMS, and 7 (from the frontal electrode) and 8 (from the temporal electrode) under temporal-TMS.

During visual WM conditions, the number of electrodes was 3 (from the frontal electrode) and 3 (from the parietal electrode) under no-TMS, 4 (from the frontal electrode) and 3 (from the parietal electrode) under frontal-TMS, and 7 (from the frontal electrode) and 8 (from the parietal electrode) under temporal-TMS. These results were almost same as the results for the analysis using the single time point data.

### 4. DISCUSSION

The present embodiment elucidated a bottom-up network in WM by measuring functional changes in theta wave phase synchronization (abbreviated as theta phase synchronization) induced by TMS. Consistent with earlier studies suggesting that theta phase synchronization reflects a global connection among relevant brain regions, theta phase synchronization was observed between WM task-relevant areas: between the frontal and parietal areas during a VWM task and between the frontal and temporal areas during an AWM task.

As expected from a previous study, which suggested that single-pulse TMS modulates global phase synchronization and information flow among brain networks at rest, TMS manipulated brain activity with global theta phase synchronization during WM tasks. EEG data of the embodiment revealed a significant difference in the amount of TMS-induced changes in theta phase synchronization between TMS-targeted areas. Additionally, TMS-induced changes in theta phase synchronization indicated that network directionality was bottom-up rather than top-down. In the parietal-TMS condition during the VWM task, induced theta phase synchronization from both frontal and parietal areas increased.

In the temporal-TMS condition during the AWM task, theta phase synchronization induced from both frontal and temporal areas increased. Although there were slight changes in theta phase synchronization in the parietal-TMS condition during the AWM task and in the temporal-TMS condition during the VWM task, these results are specific to types of modalities. Therefore, it is possible that network directionality during the WM tasks was bottom-up.

It should be noted that in the frontal-TMS condition, results from both the VWM and AWM tasks were similar to the no-TMS condition. These results show that there was no increase in induced theta phase synchronization in the frontal-TMS condition. Thus, these results indicate that network directionality during the WM tasks was not top-down. It should be noted that results in the sham-TMS condition were similar to the no-TMS condition.

These results suggest that the conclusion reached by the inventors of the present application was not influenced by auditory evoked responses induced by the “clicking” sound associated with a TMS pulse time during the experiment. Theta phase synchronization induced only increased in a case that TMS was applied to the sensory area but not the frontal lobe. Thus, it can be argued that the information network used during the task of WM was bottom-up rather than top-down.

This proposal is supported by previous findings regarding the effects of TMS on EEG signals. Previous studies have shown that TMS manipulates brain activation not only in TMS-targeted areas [12-14] but also in relevant non-TMS targeted areas [21-23]. Furthermore, a single-pulse TMS to sensory areas, but not motor areas, increases theta phase synchronization in sensory and motor areas during a resting state.

### 5. CONCLUSION

In summary, the existence of a bottom-up network in WM was clarified on the basis of the observance of increased TMS-induced theta oscillations in the frontal areas during WM tasks in the above experiment. Our approach to determine information flow by manipulating global phase synchronization would enable to evaluate network directionality of the other cognitive processing.

As described above, the construction of a system for evaluating and determining a function of the working memory that plays an important role in information processing for basic abilities of hearing, vision, language, and the like in a brain of a healthy person using EEG phase synchronization between different brain parts in TMS-EEG as an index has been confirmed.

Next, in order to construct an index for determining a pathological condition of a neuropsychiatric disorder such as depression with the same device configuration, it was applied to depressed patients as follows.

### 5. APPLICATION EXAMPLE TO EVALUATION OF DEPRESSION SEVERITY

Electro convulsive therapy (ECT), which electrically stimulates the brain of a patient, is a treatment for severe depression and intractable cases in psychiatric disorders such as severe depressive disorder and schizophrenia. Although there is clinical evidence for the efficacy of ECT, a detailed neural mechanism of a treatment is unclear. The synchronization of EEG oscillations in psychiatric patients is reported to be different from that in healthy individuals.

In order to construct an index to determine depression, comparative experiments were conducted before and after electroconvulsive therapy for transcranial magnetic stimulation (TMS) electroencephalogram or electroencephalography (EEG) during a resting state.

The inventors of the present application compared TMS-EEG in a case that the occipital lobe (visual area) before and after treatment is stimulated with respect to a depressed patient treated with electroconvulsive therapy and found the improvement of a PLV that is an index of phase synchronization of an electroencephalogram. Here, EEG data were measured from patients with depression before and after ECT during the resting state with closed eyes to investigate neural evidence for the efficacy of ECT.

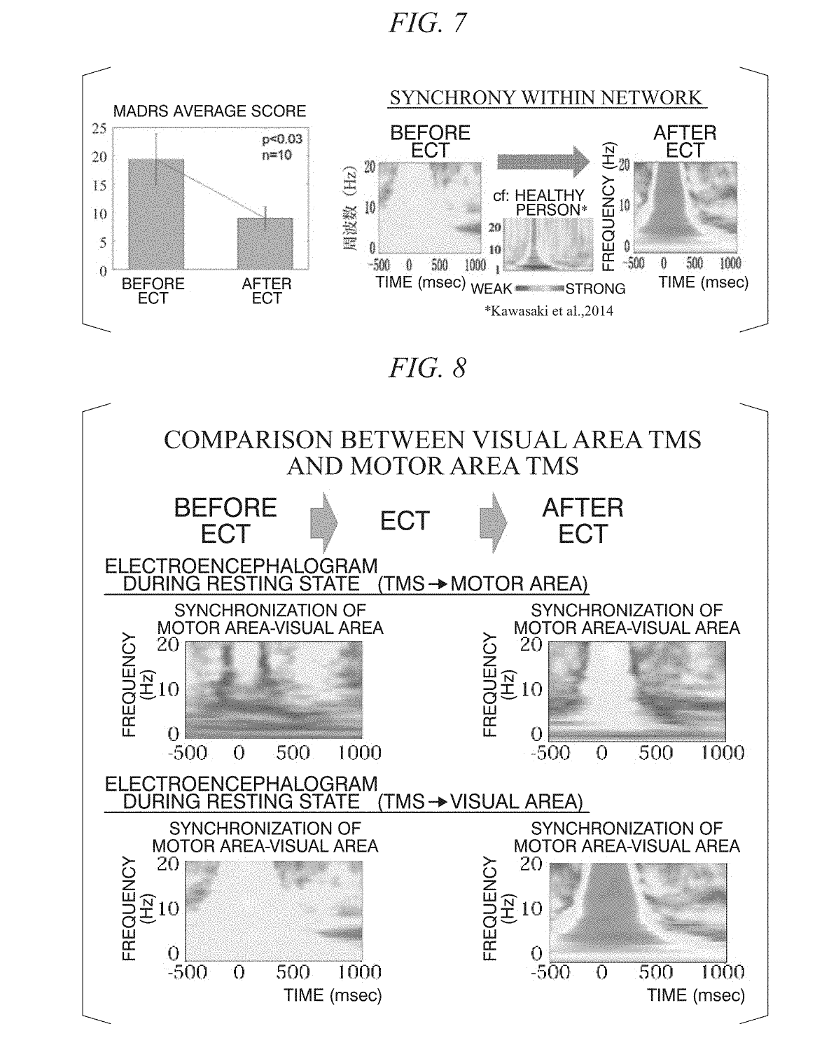

FIGS. 7 to 9 show the results of ECT performed on a subject. During EEG measurements, the brain network was modulated with TMS to a primary motor area or a primary visual brain area. Time-frequency wavelet analysis of EEG data was performed and a PLV between brain regions was calculated. In FIGS. 7 to 9, a PLV is plotted as time on the horizontal axis and frequency on the vertical axis.

FIG. 7 shows scores of a questionnaire assessment scale for depression severity (MADRS) before and after ECT and intracerebral network synchrony (PLV) before and after ECT. The higher the MADRS score, the more severe the depression. Here, during EEG measurements, the brain network was modulated with TMS to the primary visual brain region. As a result, the low frequency PLV between the visual and motor areas in the brain increased at the start of the TMS application after the ECT rather than before the ECT.

It was observed that a PLV is enhanced in a case that TMS is not applied to a motor area and the visual area is stimulated (FIG. 8). (Previous studies suggested that TMS-modulated low-frequency PLV can evaluate a relation between regions in the brain network during the resting state.)

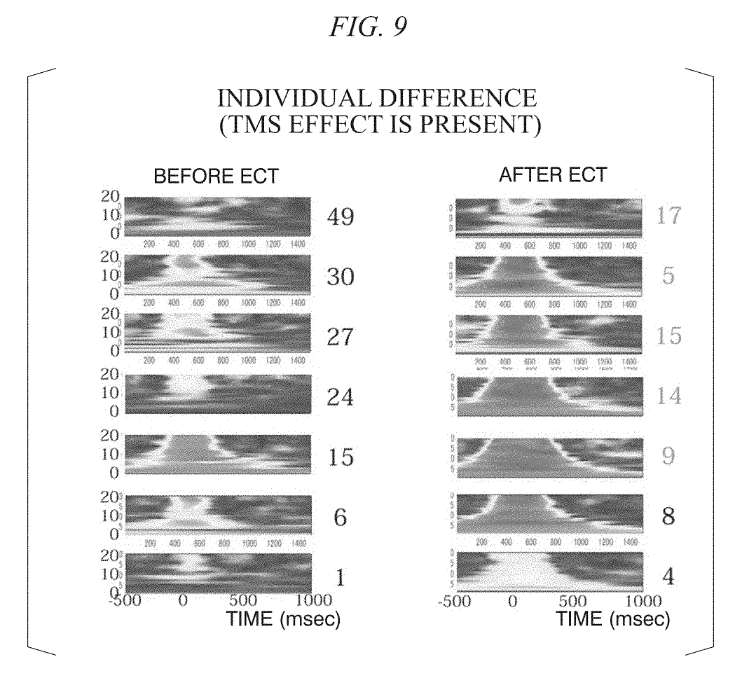

Individual differences of patients in TMS effects (improvement by ECT with respect to a PLV value of visual area TMS stimulation) are shown in FIG. 9. The PLV value is improved (phase synchrony is increased) in correspondence with the improvement (decrease) of the MADRS value after ECT (the value on the right side of the PLV plot) as compared with the value before ECT for each patient. In other words, TMS-induced EEG synchrony (PLV) represents neural evidence of the efficacy of ECT for depression. That is, it is possible to evaluate the pathological condition of neuropsychiatric disorder by detecting the EEG synchrony induced by TMS (a PLV based on a phase difference).

In summary, the inventors of the present application show that a TMS-induced PLV exhibits neural evidence of the efficacy of ECT for depression and the inventors of the present application further suggest a usage method including a TMS-induced PLV as a new method of evaluating the efficacy of ECT and other psychiatric treatments.

According to the electroencephalogram detecting device 1 described above, it is possible to evaluate a correlation between regions on the basis of reactions of a plurality of regions of a brain to different electromagnetic stimuli applied to the brain. That is, according to the electroencephalogram detecting device 1, it is possible to index a relation of corresponding parts of the brain between two points in the brain and to quantitatively evaluate a pathological condition of neuropsychiatric disorder such as depression. According to the electroencephalogram detecting device 1, it is possible to observe a treatment state of depression by measuring depressed patients over time and use an observation result as an index for selecting a treatment such as electrical stimulation therapy or drug therapy.

Although some embodiments of the present invention have been described above, these embodiments are presented as examples, and are not intended to limit the scope of the invention. These embodiments can be implemented in other various forms, and various omissions, replacements, and changes can be made within a scope not deviating from the subject matter of the invention. These embodiments and their modifications are included in the scope and the subject matter of the invention, and are included in the invention described in the claims, and the equal scope thereof.

### REFERENCE SIGNS LIST

1 Electroencephalogram detecting device
2 Electroencephalogram detecting unit
2a Cap unit
3 Computing unit
3a Electroencephalogram measuring unit
3b Electroencephalogram analyzing unit
3c Stimulus controller
4 Signal generating unit
4a Stimulus generating device
4b Coil unit
5 to 7 Fixation point
10 Red circle
12 Arrow
E Electrode
N Number
M Total
P Pulse

1. A device for evaluating a pathological condition of neuropsychiatric disorder, the device comprising:
a signal generating unit configured to apply an electromagnetic stimulus to a prescribed region in a brain of a subject;
an electroencephalogram detecting unit including a plurality of electrodes for detecting electroencephalograms in a plurality of regions in the brain in which the electromagnetic stimulus was applied to the prescribed region;
a computing unit configured to evaluate correlations between a part of the brain associated with the prescribed region to which the electromagnetic stimulus was applied and parts of the brain associated with the plurality of regions, on the basis of a plurality of electroencephalograms obtained respectively from the plurality of electrodes; and
an analysis and output unit configured to analyze and output correspondence between the correlation and severity of the neuropsychiatric disorder.

2. The device for evaluating the pathological condition of the neuropsychiatric disorder according to claim 1, wherein the computing unit computes phase synchrony between two points in the prescribed region to which the electromagnetic stimulus was applied and one region among the plurality of regions and indexes a relation of corresponding parts of the brain between the two points on the basis of the phase synchrony.

3. The device for evaluating the pathological condition of the neuropsychiatric disorder according to claim 2,
wherein the signal generating unit applies the electromagnetic stimulus to a visual area of the brain, and
wherein the computing unit indexes a relation between the visual area and another part of the brain.

4. A program for causing a computer to:
generate an electromagnetic stimulus to be applied to a prescribed region in a brain of a subject;
detect electroencephalograms in a plurality of regions of the brain using a plurality of electrodes disposed on the brain to which the electromagnetic stimulus was applied;
compute correlations between a part of the brain associated with the prescribed region to which the electromagnetic stimulus was applied and parts of the brain associated with the plurality of regions, on the basis of a plurality of electroencephalograms obtained respectively from the plurality of electrodes; and
analyze and output correspondence between the correlation and severity of neuropsychiatric disorder.

5. The device for evaluating the pathological condition of the neuropsychiatric disorder according to claim 1, wherein the neuropsychiatric disorder is depression.

6. The device for evaluating the pathological condition of the neuropsychiatric disorder according to claim 1, wherein the neuropsychiatric disorder is schizophrenia, bipolar disorder, or dementia.
